(19) United States
(12) Patent Application Publication
(10) Pub. No.: US 2016/0237390 A1
(43) Pub. Date: Aug. 18, 2016

(54) TISSUE EXTRACELLULAR MATRIX PARTICLES AND APPLICATIONS

(71) Applicant: THE JOHNS HOPKINS UNIVERSITY, Baltimore, MD (US)

(72) Inventors: Vincent Beachley, Baltimore, MD (US); Matt Gibson, Odenton, MD (US); Jennifer Elisseeff, Baltimore, MD (US)

(21) Appl. No.: 15/024,139

(22) PCT Filed: Sep. 24, 2014
(86) PCT No.: PCT/US14/57222

§ 371 Date: Mar. 23, 2016

Related U.S. Application Data
(60) Provisional application No. 61/881,856, filed on Sep. 24, 2013.

(51) Int. Cl. C12M 1/12; A61K 35/545

(52) U.S. Cl. CPC C12M 25/14; C12M 25/01; A61K 35/545

(57) ABSTRACT

An apparatus in the form of a chip is provided wherein the apparatus is prepared with decellularized extracellular matrix from various tissues and can be used to investigate the cellular interactions between the ECM and the various cell types. Three dimensional culture methods for investigating decellularized extracellular matrix from various tissues and interactions with various mammalian cell types are also provided. Methods of use of cells grown using the apparatus and methods disclosed are also provided.

### REFERENCE TO RELATED APPLICATIONS

This application claims the benefit of U.S. Provisional Patent Application No. 61/881,856, filed on Sep. 24, 2013, which is hereby incorporated by reference for all purposes as if fully set forth herein.

### BACKGROUND OF THE INVENTION

Natural extracellular matrix (ECM) materials contain an inherent microstructural and biochemical complexity that can modulate cell behaviors and tissue remodeling. This complexity is difficult to replicate in synthetic scaffolds, thus decellularized ECM matrices are recognized as an attractive tool in regenerative medicine. Different types of tissue have different properties. Accordingly, it has been shown that decellularized ECM generated from different types of tissue elicit unique cellular responses in vitro.

However, to date, there has not been available methods for systematically testing cell behaviors and tissue remodeling on decellularized ECM derived from multiple tissue types in an efficient and cost-effective manner.

### SUMMARY OF THE INVENTION

In accordance with some embodiments, the present invention provides a novel chip-type apparatus which can be used to investigate the interactions of decellularized extracellular matrix (DECM) from various mammalian tissues with many mammalian cell types including stem cells. In some other embodiments, the present invention provides novel processes for preparation of three dimensional culture of mammalian cells with DECM and use of the processes for making spheroids or spheroid aggregates of mammalian cells and DECM. Methods of use of the apparatus and culturing processes for in vitro analysis of cellular interactions and use in treatment of certain conditions in vivo are also provided.

In accordance with an embodiment, the present invention provides an apparatus for culturing cells comprising: a first functionalized substrate having at least one functionalized surface, a gel pad disposed on the functionalized surface of the first functionalized substrate, an array comprising a plurality of discrete layers of collagen positioned on top of the gel pad, each having a defined area and being substantially aligned with one another and defining a space between one another, and a layer of decellularized extracellular matrix particles (DECM) positioned on top of at least one or more of the discrete layers of collagen, wherein the DECM is capable of supporting cellular growth.

In accordance with another embodiment, the present invention provides a process for forming spheroid aggregates of mammalian stem cells and decellularized extracellular matrix (DECM) particles comprising: a) preparing a solution of DECM particles in a suitable growth media, b) preparing a solution of mammalian stem cells in the same suitable growth media, c) preparing a mixture of a) and b) at a ratio in a range of 10:1 to 1:10 v/v DECM particle suspension:mammalian cells, d) suspending the mixture of c) in a hanging drop culture for a period of between 2 days and 7 days, e) replacing the growth media with a suitable induction media, and f) allowing the culture to grow for period of time sufficient to produce a spheroid aggregate comprising mammalian stem cells and DECM.

In accordance with a further embodiment, the present invention provides a spheroid aggregate of mammalian stem cells and DECM particles made using the described above.

In accordance with an embodiment, the present invention provides a method for identifying the interaction of mammalian stem cells with differing types of extracellular matrix in vitro comprising: a) preparing an apparatus described above, or the method of forming spheroid aggregates described above, with DECM particles from one or more different tissues, b) obtaining a sample of mammalian stem cells of interest, c) placing a sufficient amount of the cells of interest of a) in the apparatus with suitable growth media, d) culturing the mammalian stem cells of interest for a sufficient period of time, and f) comparing the effect of the DECM particles from one or more different tissues on the growth of the mammalian stem cells of interest.

In accordance with still another embodiment, the present invention provides a method of implanting spheroid aggregates of mammalian stem cells and decellularized extracellular matrix (DECM) particles in a subject comprising: a) identifying a subject in need of spheroid aggregates of mammalian stem cells and decellularized extracellular matrix (DECM) particles, b) identifying a site in the subject in need of implantation of spheroid aggregates of mammalian stem cells and decellularized extracellular matrix (DECM) particles, and c) implanting the spheroid aggregates of mammalian stem cells and decellularized extracellular matrix (DECM) particles in the subject at the identified site.

### BRIEF DESCRIPTION OF THE DRAWINGS

FIG. 1 (A) Depicts fabrication of the spotted ECM array embodiment of the present invention. (i) Fresh tissue is decellularized, processed into powder, and suspended in water. (ii) Acrylamide coated cover slips are first spotted with collagen I solution and allowed to dry. ECM suspension is spotted on the dried collagen I spots and allowed to dry. (iii) A silicone gasket with 3 mm holes is used to precisely pattern 40 individual spots per cover slip. (B) A photograph of a spotted cover slip stained by H&E. Here decellularized ECM from 8 different tissue sources are spotted with 3 replicates. (C) Photomicrographs showing microstructure and composition of ECM spots. Staining for total protein (red), collagen I (green), and fibronectin (yellow) are overlaid on the right

FIG. 2(A) (i) Depicts the agglomeration of cells and ECM particles into a cell/tissue spheroid in hanging drop culture depicted in a schematic. (ii) Images show the progression of formation of a cell/tissue spheroid containing hASC cells and decellularized cartilage particles. (C) Cell/tissue spheroids made with ASC cells are shown stained with Masson's trichrome. Cell=red, collagen=blue.

FIG. 3 (Left, 2D) ASC cells cultured for 6 days in osteogenic induction (OM) or control (growth media, GM) media conditions. (i) Calcien AM staining shows cell density and morphology and alizarin red staining labels calcified matrix deposition. (ii) The percent area of each spot positively stained for alizarin red is quantified (n=9), OM=blue, GM=red. (iii) An embodiment of the apparatus of the present invention incubated in GM is shown on the left and one incubated in OM on the right. (Right, 3D) (i) Cell/tissue spheroids containing ASC cells and ECM particles are stained with alizarin red after 7 (OM) or 14 days (GM). (ii) A sample slide stained with alizarin red showed the microarray of the apparatus used for histological processing.

### DETAILED DESCRIPTION OF THE INVENTION

As used herein, the term “decellularized extracellular matrix (DECM)” means homogenizing or mincing the tissue and manipulating the tissue with a buffer to promote lipid and cell removal to prepare decellularized tissue. The buffers used can be any suitable buffer including phosphate buffered saline (PBS). Agents to promote decellularization can include one or more of a weak acid, such as a weak organic acid, a non-ionic detergent, and a bile acid. After treatment of the tissue with a buffer or agent not at or about physiological pH, a buffer to adjust pH of the tissue to physiological pH. Decellularization can also include nuclease treatment of the material using known enzymes and agents to remove nucleic acids.

The source of the DECM tissue is mammalian tissue. The mammalian tissue can be obtained from any mammal, most conveniently from larger mammals to provide sufficient starting material.

In some embodiments, DECM tissue is lyophilized and then cryogenically pulverized in a cryomill at −195° C. under liquid nitrogen. The resulting powder is suspended in distilled water or suitable growth media and sonicated with a probe sonicator in an ice bath. The resulting suspension is centrifuged at 14000 rpm for a sufficient period of time and resuspended in DI water to remove any residual reagents left over from decellularization. The result is a DECM particulate suspension which can be filtered.

The DECM apparatus and methods of the present invention can be used to grow and/or deliver various types of living cells (e.g., a mesenchymal stem cells, cardiac stem cells, liver stem cells, retinal stem cells, and epidermal stem cells). As used herein, the term “mammalian stem cells” means without limitation, a cell that gives rise to a lineage of progeny cells. Mesenchymal stem cells may not be differentiated and therefore may differentiate to form various types of new cells due to the presence of an active agent or the effects (chemical, physical, etc.) of the local tissue environment. Examples of mesenchymal stem cells include osteoblasts, chondrocytes, and fibroblasts. For example, osteoblasts can be delivered to the site of a bone defect to produce new bone; chondrocytes can be delivered to the site of a cartilage defect to produce new cartilage; fibroblasts can be delivered to produce collagen wherever new connective tissue is needed; neurectodermal cells can be delivered to form new nerve tissue; epithelial cells can be delivered to form new epithelial tissues, such as liver, pancreas etc.

By “hydrogel” is meant a water-swellable polymeric matrix that can absorb water to form elastic gels, wherein “matrices” are three-dimensional networks of macromolecules held together by covalent or noncovalent crosslinks. On placement in an aqueous environment, dry hydrogels swell by the acquisition of liquid therein to the extent allowed by the degree of cross-linking.

“Treating” or “treatment” is an art-recognized term which includes curing as well as ameliorating at least one symptom of any condition or disease. Treating includes reducing the likelihood of a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing any level of regression of the disease; inhibiting the disease, disorder or condition, e.g., impeding its progress; and relieving the disease, disorder or condition, even if the underlying pathophysiology is not affected or other symptoms remain at the same level.

“Prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

As used herein, the term “surfactant” refers to organic substances having amphipathic structures, namely, are composed of groups of opposing solubility tendencies, typically an oil-soluble hydrocarbon chain and a water-soluble ionic group. Surfactants can be classified, depending on the charge of the surface-active moiety, into anionic, cationic and nonionic surfactants. Surfactants often are used as wetting, emulsifying, solubilizing and dispersing agents for various pharmaceutical compositions and preparations of biological materials.

An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

Cross-linked herein refers to a composition containing intermolecular cross-links and optionally intramolecular cross-links, arising from, generally, the formation of covalent bonds. Covalent bonding between two cross-linkable components may be direct, in which case an atom in one component is directly bound to an atom in the other component, or it may be indirect, through a linking group. A cross-linked gel or polymer matrix may, in addition to covalent, also include intermolecular and/or intramolecular noncovalent bonds such as hydrogen bonds and electrostatic (ionic) bonds.

“Functionalized” refers to a modification of an existing molecular segment or group to generate or to introduce a new reactive or more reactive group (e.g., imide group) that is capable of undergoing reaction with another functional group (e.g., an amine group) to form a covalent bond. For example, carboxylic acid groups can be functionalized by reaction with a carbodiimide and an imide reagent using known procedures to provide a new reactive functional group in the form of an imide group substituting for the hydrogen in the hydroxyl group of the carboxyl function.

“Gel” refers to a state of matter between liquid and solid, and is generally defined as a cross-linked polymer network swollen in a liquid medium. Typically, a gel is a two-phase colloidal dispersion containing both solid and liquid, wherein the amount of solid is greater than that in the two-phase colloidal dispersion referred to as a “sol.” As such, a “gel” has some of the properties of a liquid (i.e., the shape is resilient and deformable) and some of the properties of a solid (i.e., the shape is discrete enough to maintain three dimensions on a two-dimensional surface).

Hydrogels consist of hydrophilic polymers cross-linked to from a water-swollen, insoluble polymer network. Cross-linking can be initiated by many physical or chemical mechanisms. Photopolymerization is a method of covalently crosslink polymer chains, whereby a photoinitiator and polymer solution (termed “pre-gel” solution) are exposed to a light source specific to the photoinitiator. On activation, the photoinitiator reacts with specific functional groups in the polymer chains, crosslinking them to form the hydrogel. The reaction is rapid (3-5 minutes) and proceeds at room and body temperature. Photoinduced gelation enables spatial and temporal control of scaffold formation, permitting shape manipulation after injection and during gelation in vivo. Cells and bioactive factors can be easily incorporated into the hydrogel scaffold by simply mixing with the polymer solution prior to photogelation.

Cross-linked polymer matrices used in the present invention may include and form hydrogels. The water content of a hydrogel may provide information on the pore structure. Further, the water content may be a factor that influences, for example, the survival of encapsulated cells within the hydrogel. The amount of water that a hydrogel is able to absorb may be related to the cross-linking density and/or pore size. For example, the percentage of imides on a functionalized macromer, such as chondroitin sulfate, hyaluronic acid, dextran, carboxy methyl starch, keratin sulfate, or ethyl cellulose, may dictate the amount of water that is absorbable.

The gels used in the present invention may comprise monomers, macromers, oligomers, polymers, or a mixture thereof The polymer compositions can consist solely of covalently crosslinkable polymers, or ionically crosslinkable polymers, or polymers crosslinkable by redox chemistry, or polymers crosslinked by hydrogen bonding, or any combination thereof The reagents should be substantially hydrophilic and biocompatible.

As used herein, the term “gel pad” means a hydrogel made of cross-linked acrylamide and bis-acrylamide. It is understood by those of skill in the art, that other polymers can be used that are biocompatible.

Buffering agents help to maintain the pH in the range which approximates physiological conditions. Buffers are preferably present at a concentration ranging from about 2 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts, such as Tris, HEPES and other such known buffers can be used.

Non-ionic surfactants or detergents (also known as “wetting agents”) may be used in the preparation of DECM, without causing denaturation of the proteins. Suitable non-ionic surfactants include polysorbates (20, 80 etc.), polyoxamers (184, 188 etc.), Pluronic® polyols and polyoxyethylene sorbitan monoethers (TWEEN-20®, TWEEN-80® etc.).

The formulations to be used for in vivo administration must be sterile. That can be accomplished, for example, by filtration through sterile filtration membranes. For example, the formulations of the present invention may be sterilized by filtration.

The spheroid aggregates of the present invention will be formulated, dosed and administered in a manner consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the spheroid aggregates to be administered will be governed by such considerations, and can be the minimum amount necessary to prevent, ameliorate or treat a disorder of interest. As used herein, the term “effective amount” is an equivalent phrase refers to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease. For example, a treatment of interest can increase the use of a joint in a host, based on baseline of the injured or diseases joint, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In another embodiment, an effective amount of a therapeutic or a prophylactic agent of interest reduces the symptoms of a disease, such as a symptom of arthritis by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. Also used herein as an equivalent is the term, “therapeutically effective amount.”

Cells dissociated from a variety of tissues, mostly of embryonic origin, have been demonstrated to be capable under appropriate experimental conditions to re-assemble into aggregates resembling the organization and architecture of their tissue of origin. In such structures where no particular geometry is imposed to the cells and where cell-cell contacts are maximized, the cells survived longer while maintaining their differentiated functions and, often, continuing their normal differentiation. Indeed, a number of studies making use of the three-dimensional re-aggregate or spheroid culture system have suggested that cells may require a proper three-dimensional cyto-architecture as found in vivo for optimal functioning. As used herein, the terms “spheroids or spheroid aggregates” means a three dimensional aggregate of a mammalian cell or stem cell with one or more types of DECM. Other components and biological agents can also be included in the spheroids of the present invention.

While not wishing to be bound to any particular method, the spheroids of the present invention can be made using any cell culture methods which allows cellular aggregates and spheroids to form. In an embodiment, the method of preparing spheroid aggregates of the present invention comprises the use of hanging drop culture methods.

EXAMPLES

Tissue decellularization. Porcine tissues were harvested from 6 month old market weight pigs weighing approximately 100 kg (Wagner's Meats, Mt. Airy, Md.) and frozen at −20° C. Tissue was thawed and cut into pieces approximately 100 mm³ and rinsed several times with phosphate buffered saline (PBS). Bone tissue required an addition decalcification preparation in 10% formic acid for 18 hours in room temperature and fat was mechanically pressed to reduce lipid content before decellularization. Tissue was decellularized by incubation with three different solutions with thorough washing in PBS between each step: (1) 3% peracetic acid for 3 hours at 37° C., (2) 1% Triton™ X-100 containing 2 mM EDTA for 18 hrs at 37° C., (3) 600 U/mL DNAse containing 10 mM MgCl₂ for 18 hours at 37° C. After the final treatment the tissue was washed thoroughly with PBS followed by distilled water and then lyophilized.

Decellularized tissue suspensions. Lyophilized decellularized tissue was cryogenically pulverized in a cryomill (SPEX 6770, SPEX SamplePrep®, Metuchen, N.J.) at −195° C. under liquid nitrogen. The resulting powder was suspended in distilled water or DMEM media at 10 mg/ml and sonicated with a probe sonicator (GE 130PB, Cole Parmer) at an output power of 10-15W two times for 30 seconds in an ice bath. The suspension was centrifuged at 14000 rpm for 10 minutes and resuspended in DI water to remove any residual reagents left over from decellularization. Sonication was repeated and the suspension was filtered through a 40 μm cell sieve. The final concentration was determined by lyophilizing aliquots.

Chip preparation. Glass cover slips (22×60 mm) were cleaned and functionalized with methacrylate groups as previously described (Stem Cells Dev. 2008;17(1):29-39). Acrylimide was mixed with bis-acrylimide and dissolved in DI water at a concentration of 10.55% and 0.55% wt/v respectively. A photointiatior solution of Igracure (12959) dissolved in methanol at 200 mg/ml was added to the acrylimide solution at a concentration of 10% v/v. An acrylimide gel pad was fixed to the functionalized coverslip by polymerizing the working solution with ultraviolet (UV) light. A 20 μL drop of working solution was pipetted on the functionalized 22×60 mm coverslip and an untreated 22×50 mm glass slide was carefully placed on top of the liquid to form a thin layer estimated to be 18 μL thick. The solution was polymerized for 10 minutes and the 22×50 mm coverslip was removed after incubation in DI water for 30 minutes. Gel coated slides were soaked in DI water overnight dried on a hot plate at 40° C. for 45 minutes.

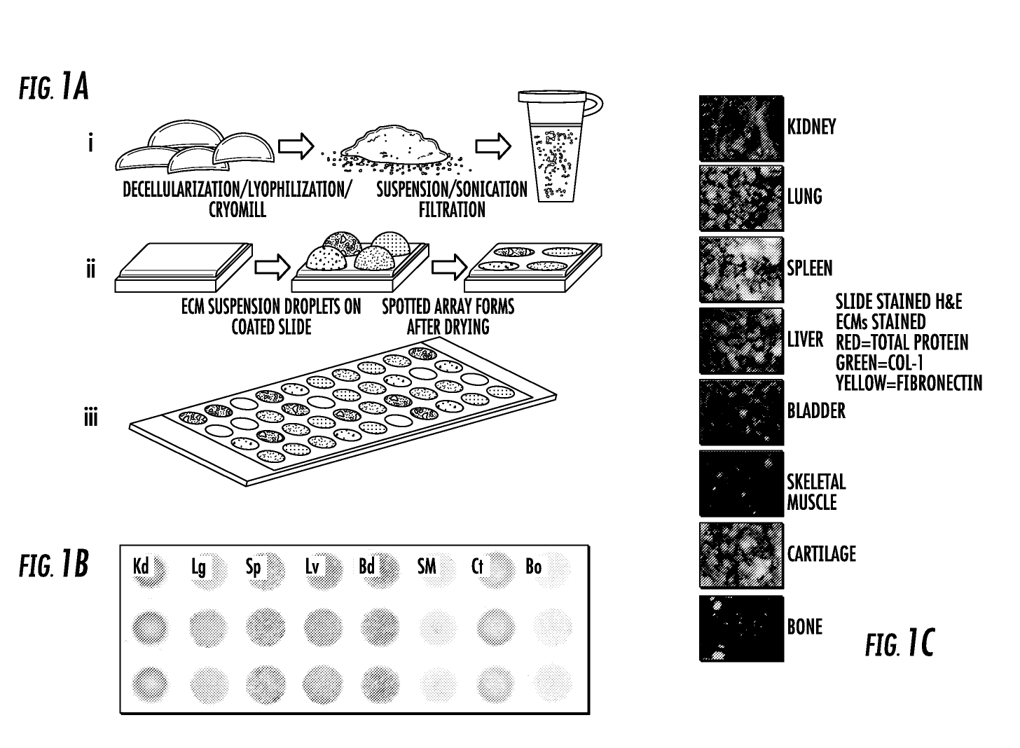

Silicon gaskets with arrays 3 mm diameter wells (Grace Biolabs, CWCS-50R) were placed on the dry gel coated slide with 40 wells in full contact. 9 μL of collagen (Sigma, C7661) dissolved at 0.25 mg/ml in 0.1M acetic acid, was pipetted in each chamber and allowed to dry overnight. Next, 10 μL of DECM suspension was spotted in each of the collagen coated wells. The concentration of each type of DECM (1-3 mg/ml) was determined by the concentration required to form a complete monolayer of DECM on the chip. These concentrations were previously determined by spotting each type of DECM from a concentration gradient (data not shown). Spotted chips were left to dry overnight in a cell culture hood at room temperature and the gaskets were removed. Chips were sterilized with UV light for 30 minutes on each side. A schematic depicting fabrication of the apparatus is shown in FIG. 1.

Cell culture. Human adipocyte stem cells (hASC) cells were isolated as previously described (Stem Cells. 2006;24(2):376-85) and passaged at 90% confluence in growth media. hASC cells were cultured in growth media (GM, Dulbecco's Modified Eagle Medium (Invitrogen 11965, DMEM) supplemented with 10% fetal bovine serum (FBS), 1% penicillin streptomycin (P/S)), and for some experiments, osteogenic differentiation induction media (OM, DMEM, 10% FBS, 1% P/S, 100 nM dexamethasone, 50 μM ascorbic acid-2-phosphate, 10 mM β-glycerophosphate).

For preparation of cell culture in the chip apparatus of the present invention, cells were suspended in 8 mL of culture media and seeded on spotted chips in 4 well rectangular plates (NUNC) at 6000 cells/cm². hASCs were cultured to confluence in GM for 5 days and then media was switched to indicated induction media. Media was changed at 24 hours after seeding and every three days after.

hASC spheroids were cultured using 96 well Gravity Plus hanging drop culture plates (insphero). DECM particles suspensions were diluted to 0.8 mg/ml in serum free DMEM culture media and hASC cells were suspended in GM at about 850,000 cells/ml. 40 μL of a 1:1 mixture of DECM particle suspension and hASC cell suspension was pipetted into the plate for form hanging drops. Media was changed with GM every 2 days. After 6 days of culture, the resulting spheroids were moved Gravity Trap plates (insphero) and media was replaced with indicated induction media. Media was then changed every 3 days.

Histology. To characterize the various effects that DECM has on cells in culture, the chips were washed with water and stained with hemotoxilyn and eosin (H&E), masons trichrome, and immunostained against antibodies to total protein, collagen I, and fibronectin.

Chips seeded with hASC cells were imaged for and calcified matrix content was stained. Just prior to harvest, live cells were stained with calcien AM and images were taken. Chips were then washed with PBS and fixed for 20 minutes in 4% paraformaldehyde. Chips were washed toughly with DI water and incubated with alizarin red solution (pH 4.1) for 25 minutes. Chips were then briefly rinsed with DI water 3 times and then a fourth time for 5 minutes, before dehydration in acetone, acetone:xylene (50:50), and xylene, followed by addition of a cover slip. Slides were imaged using a slide scanner and the % area stained was quantified using adjusting the color threshold in Image J.

At time of harvest spheroids were washed with PBS and fixed for 1 hour in 4% paraformaldehyde. Spheroid sections were stained with H&E, masson's trichrome to assess cell/ECM organization and collagen content. Calcium was stained with alizarin red for 5 minutes, followed by brief rinsing in acetone, acetone:xylene, and xylene. Slides were imaged at 20× with a slide scanner.

### EXAMPLE 1

Two dimensional chip characterization. The chip of the present invention with spotted arrays of DECM stained with hemotoxylin and eosin is shown in FIG. 1B. Differences in total protein, fibronectin, and collagen I content can be seen in FIG. 1C. The microstructure of each tissue DECM spot varied for each tissue

### EXAMPLE 2

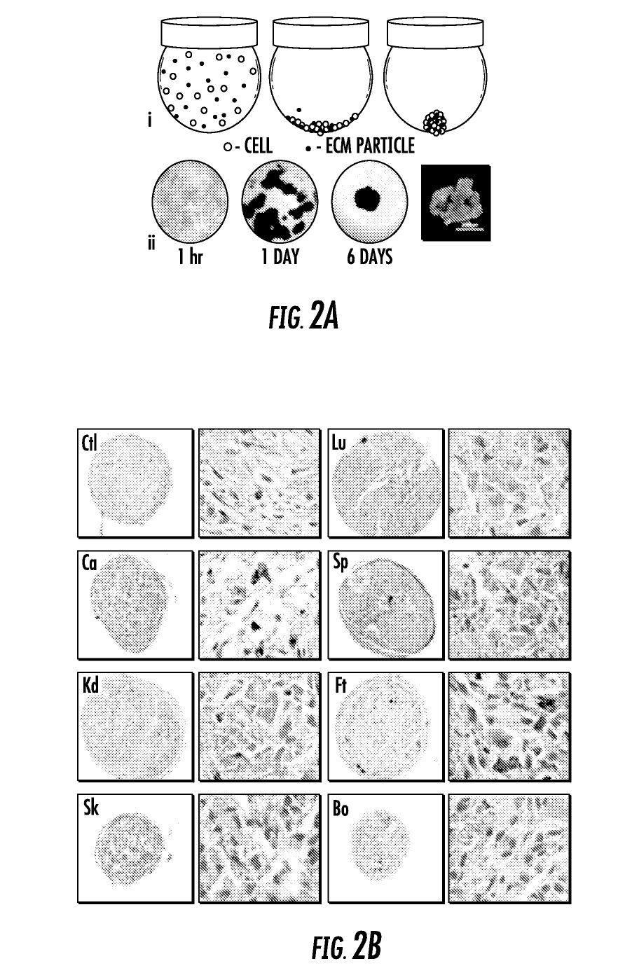

Three dimensional culture characterization. Cells and DECM particles were aggregated at the bottom of the hanging drops to form cell/DECM particle spheroids. After one day small aggregates had formed, and after 6 days the smaller aggregates fused into large single spheroids (FIG. 2). A mold embedding system allowed sectioning of up to 40 spheroids in one block, and single sections were produced that contained up to 90% of the embedded spheroids. Generally cells and DECM particles adopted a well distributed arrangement within the spheroids. In many cases it appeared that a layer of cells wrapped around the outer shell of the cell/particle interior. Spheroids containing hASC cells and several different DECM tissue types are shown stained with Masson's trichrome in FIG. 2.

### EXAMPLE 3

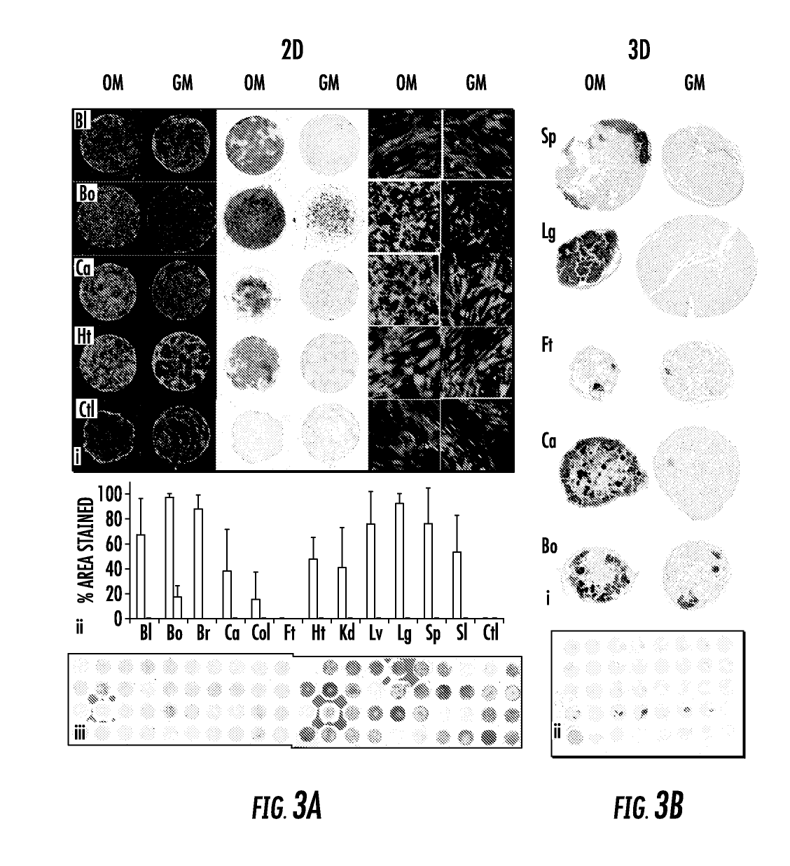

hASC cell interactions with DECM on chip. Adipose derived stem cells attached and proliferated on all 13 DECM substrates and collagen-I controls. Limited cell attachment on the acylamide gel intermediate space was observed, but most of these cells died off after a few days. After 5 days of culture in growth media, confluent or near confluent monolayers of hASC cells were formed on all spot types. After 6 days of culture in induction media, some cell monolayers began to peel from the DECM spots. Most notable were brain and heart tissue, and soluble collagen control, in GM, and soluble collagen control in osteogenic media. Cells adopted various morphologies on the different DECMs as shown in FIG. 3. Cell morphology appeared to be highly dependent on DECM type, with less influence from the media type. Cells cultured in osteogenic media differentiated into a bone lineage as confirmed by deposition of calcified matrix. Alizarin red staining was generally confined to the DECM spots, but some highly geometric staining was observed between spots for unknown reasons. Positive alizarin red staining for calcified matrix was strongly dependent on tissue type in with the highest positive staining after 6 days in OM approaching 100% total area on bone DECM and 0% on Fat DECM and soluble collagen control spots. Alizarin red staining was only present on bone DECM spots for cells cultured in growth media. A strong correlation between morphology and calcified matrix was not observed. Alizarin red staining for calcified matrix at 6 days after OM induction was quantified for % area positively stained (n=9, FIG. 3).

### EXAMPLE 4

hASC cell interactions with DECM in 3D cultures. Calcium deposition was also present within 3D hASC/tissue particles spheroids. Alizarin red staining on spheroids incubated in OM for 7 days, or GM for 14 days after formation, and is shown in FIG. 3. Similar to results seen in on 2D chips, alizarin red staining was strong for constructs cultured in OM, and positive staining was only present in constructs with bone ECM when cultured in GM. The propensity of each tissue type for calcium matrix deposition in 3D spheroids was similar to what was seen in 2D, with the exception that hASC/bone ECM spheroids demonstrated less positive staining compared to lung and cartilage tissue.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An apparatus for culturing cells comprising:
a first functionalized substrate having at least one functionalized surface;
a gel pad disposed on the functionalized surface of the first functionalized substrate;
an array comprising a plurality of discrete layers of collagen positioned on top of the gel pad, each having a defined area and being substantially aligned with one another and defining a space between one another; and
a layer of decellularized extracellular matrix particles (DECM) positioned on top of at least one or more of the discrete layers of collagen, wherein the DECM is capable of supporting cellular growth.

2. The apparatus of claim 1 wherein the functionalized substrate is glass.

3. The apparatus of claim 1, wherein the gel pad comprises a polymerized biocompatible gel.

4. The apparatus of claim 1, wherein the discrete layers of collagen comprise collagen I.

5. The apparatus of claim 1, wherein the discrete layers of collagen are circular in shape.

6. The apparatus of claim 5, wherein the discrete layers of collagen have a diameter of between 1 to 10 mm.

7. The apparatus of any of claim 1, wherein the DECM particles are derived from one or more different tissues.

8. The apparatus of claim 7, wherein the tissues are selected from the group consisting of kidney, lung, liver, spleen, bladder, skeletal muscle, cartilage, bone, heart, intestine, tendon, and brain.

9. The apparatus of claim 1, wherein the DECM particles are derived from one or more different species of mammal.

10. A process for forming spheroid aggregates of mammalian stem cells and decellularized extracellular matrix (DECM) particles comprising:
a) preparing a suspension of DECM particles in a suitable growth media;
b) preparing a suspension of mammalian stem cells in the same suitable growth media;
c) preparing a mixture of a) and b) at a ratio in a range of 10:1 to 1:10 v/v DECM particle solution:mammalian cells;
d) suspending the mixture of c) in a hanging drop culture for a period of between 2 days and 7 days;
e) replacing the growth media with a suitable induction media; and
f) allowing the culture to grow for period of time sufficient to produce a spheroid aggregate comprising mammalian stem cells and DECM.

11. The process of claim 10, further comprising the addition of one or more growth factors or cytokines to the media of e).

12. The process of claim 10, wherein the DECM particles are derived from one or more different tissues.

13. The process of claim 12, wherein the tissues are selected from the group consisting of kidney, lung, liver, spleen, bladder, skeletal muscle, cartilage, bone, heart, intestine, tendon, and brain.

14. The process of claim 10, wherein the DECM particles are derived from one or more different species of mammal.

15. The process of claim 10, wherein the mammalian stem cells are selected from the group consisting of adipose stem cells, mesenchymal stem cells, cardiac stem cells, hepatic stem cells, retinal stem cells, and epidermal stem cells.

16. A spheroid aggregate of mammalian stem cells and DECM particles made using the process of claim 10.

17. A method for identifying the interaction of mammalian stem cells with differing types of extracellular matrix in vitro comprising:
a) preparing an apparatus of claim 1, or using the process of claim 10, with DECM particles from one or more different tissues;
b) obtaining a sample of mammalian stem cells of interest;
c) placing a sufficient amount of the cells of interest of a) in the apparatus claim 1 with suitable growth media, or using the process of claim 10;
d) culturing the mammalian stem cells of interest for a sufficient period of time; and
f) comparing the effect of the DECM particles from one or more different tissues on the growth of the mammalian stem cells of interest.

18. A method of implanting spheroid aggregates of mammalian stem cells and decellularized extracellular matrix (DECM) particles in a subject comprising:
a) identifying a subject in need of spheroid aggregates of mammalian stem cells and decellularized extracellular matrix (DECM) particles;
b) identifying a site in the subject in need of implantation of spheroid aggregates of mammalian stem cells and decellularized extracellular matrix (DECM) particles; and
c) implanting the spheroid aggregates of mammalian stem cells and decellularized extracellular matrix (DECM) particles in the subject at the identified site.

19. The method of claim 18, wherein the spheroid aggregates of mammalian stem cells and decellularized extracellular matrix (DECM) particles are made using the process of claim 10.
